(19) United States
(12) Patent Application Publication
(10) Pub. No.: US 2008/0103040 A1
(43) Pub. Date: May 1, 2008

(54) Catalytic Composition for the Insertion of Carbon Dioxide Into Organic Compounds

(76) Inventors: Mercedes Alvaro Rodriguez, Valencia (ES); Esther Carbonell Llopis, Valencia (ES); Avelino Corma Canos, Valencia (ES); Hermenegildo Garcia Gomez, Valencia (ES)

Correspondence Address: WENDEROTH, LIND & PONACK, L.L.P., 2033 K STREET N. W., SUITE 800, WASHINGTON, DC, 20006-1021, US

(21) Appl. No.: 11/660,995

(22) PCT Filed: Aug. 24, 2005
(86) PCT No.: PCT/ES05/70124

§ 371 Date: Sep. 13, 2007

(30) Foreign Application Priority Data
Aug. 25, 2004 (ES) P200402112

(51) Int. Cl. B01J 31/12; C07C 68/04

(52) U.S. Cl. 502/87; 502/150; 502/159; 502/171; 502/300; 502/319; 502/325; 502/338; 502/353; 502/355; 558/277

(57) ABSTRACT

The invention relates to a catalytic composition comprising: a first component which is at least a component with one or more metals from groups 3A, 4A, 5A, 6A, 7A, 8, 1B, 2B, 3B, 4B; and a second component selected from (1) at least one ionic liquid which consists of a compound formed by cations and anions and which is a liquid at ambient temperature, (ii) a matrix to which the first component is bound or on which it is supported, and (iii) a combination of the two. The invention relates to the use of said catalytic composition in a method for the insertion of carbon dioxide into an organic compound and, preferably, a compound selected from epoxides, acetals and orthoesters. The invention also relates to catalytic compositions comprising said metallic compounds.

### STATE OF THE ART PRIOR TO THE INVENTION

There currently exists great interest in developing industrial processes which use carbon dioxide as raw material. On the one hand, CO₂ is considered to be a renewable raw material which presents advantages over methane as a source of industrial compounds with one carbon atom. On the other hand, processes which consume atmospheric CO₂ can help to comply with the Kyoto agreements with regard to climate change, thereby helping to compensate for CO₂ emissions.

One reaction which has been described in the literature is the insertion of CO₂ into epoxides in order to give cyclic carbonates (equation 1). The precedents so far have used complexes of chromium and cobalt as salen ligands and phthalocyanines of chromium, in which none of the catalysts described are reusable.

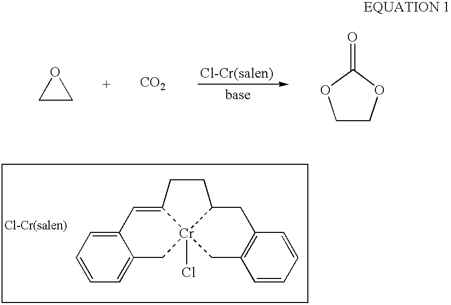

Moreover, searches that have been conducted by the inventors have not revealed any precedents in the chemical literature on the reaction of acetals or orthoesters with carbon dioxide. In this case, the products that are formed are linear carbonates and the corresponding ketone (equation 2). The driving force which shifts the equilibrium, making the process thermodynamically possible, is the formation of a carbonyl group simultaneously with the reaction of the CO₂.

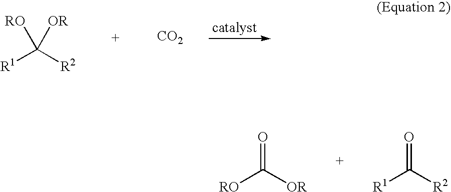

The carbonates can have applications as gasoline additives in order to improve the octane rating, as alternative industrial solvents to volatile organic solvents and as starting reagents in alkylation and carboxyalkylation reactions in substitution for halogenated compounds.

### DESCRIPTION OF THE INVENTION

The present invention relates to catalytic systems containing a metal salt and a metal complex which acts as a Lewis acid, which can be recovered after the reaction and be reused, in addition to permitting a continuous process to be designed for carrying out the transformations defined by equations 1 and 2. One of the systems operates in the homogeneous phase and uses an ionic liquid as solvent, while the other system, also the object of this invention, operates in the heterogeneous phase and the catalyst is a solid which remains insoluble during the reaction.

The reusable homogeneous system is based on the use of an ionic liquid containing a base in which is dissolved the salt or metal complex. An ionic liquid is understood to be any salt whose cation is organic and which presents a liquid state at room temperature. The reaction products are separated from the ionic phase by any procedure of physical separation such as for example extraction with a solvent that is immiscible with the ionic liquid such as hexane or ethers. Alternatively, the volatile products can be collected from the ionic liquid by evaporation. As ionic liquids, use can be made of those having an imidazolium structure substituted in the 2 position of the ring or not and with different counter anions, two possible anions being hexafluorophosphate and tetrafluoroborate. Structure 1 corresponding to 1-butyl-3-methylimidazolium hexafluorophosphate represents a possible example.

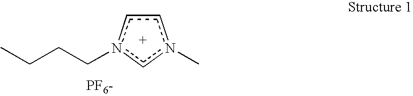

As counter anion, use can also be made of acidic anions of aluminium or other Lewis acid prepared by the simple dissolution of two equivalents of the Lewis acid with the chloride of the ionic liquid under anhydrous conditions. An example is the anion Al₂Cl₇⁻, resulting from the reaction of two equivalents of anhydrous aluminium trichloride with the chloride of an organic cation of the imidazolium type (Eq. 3).

2AlCl₃+Cl⁻→Al₂Cl₇⁻ (Eq. 3)

With regard to the metal complexes, in addition to complexes of Schiff type bases, these can be modified in order to adapt them to the ionic liquid, improving their partition coefficient and minimising losses of catalyst during the recovery of the products, introducing an imidazolium substituent as described in Scheme 2 for the case of the salen aluminium complex.

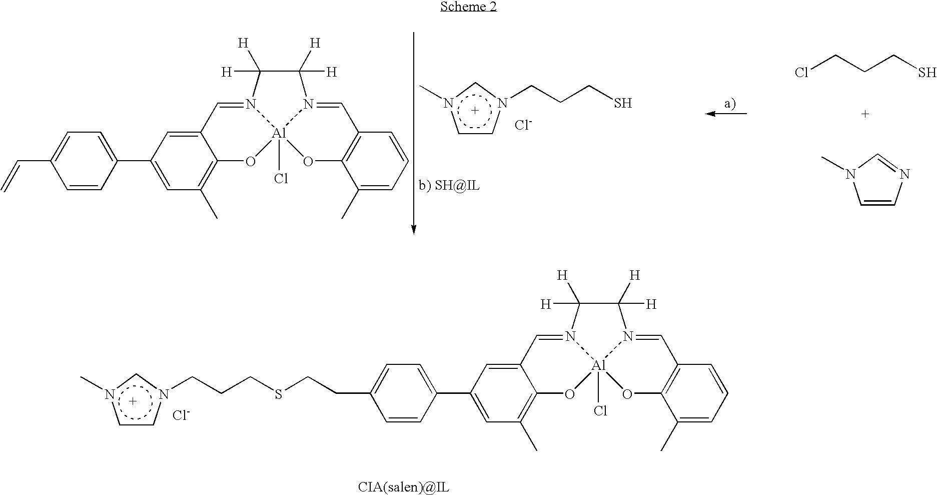

The metal complexes can be chiral when asymmetric carbons are introduced into the ligand. In these cases, the chiral complex can, for equation 1, insertion of CO₂ into epoxides, shown above, induce the formation of cyclic carbonates with enantiometric excess.

Alternatively, the ionic liquid, another organic solvent and in particular the diethyl carbonate can be used as the medium when a metal salt or metal complex is used supported on a polymeric or inorganic solid of large surface area. The metal salt or complex can also be dispersed in any activated carbon, graphite or other allotropic form of carbon.

A procedure by which a salen complex can be anchored consists of modifying the ligand by means of the introduction of peripheral chloromethyl groups which act as reactive groups in order to carry out the bonding with the solid previously functionalised as described in Schemes 3 and 4.

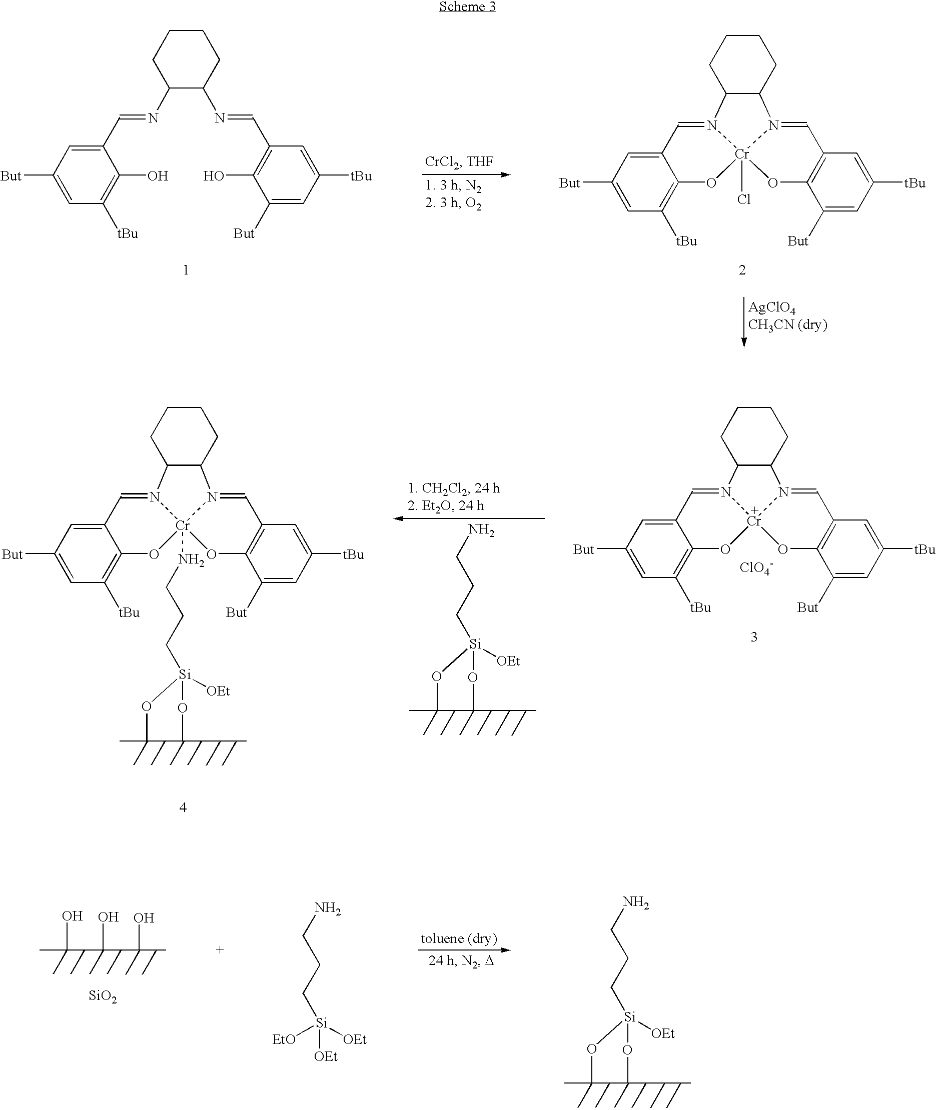

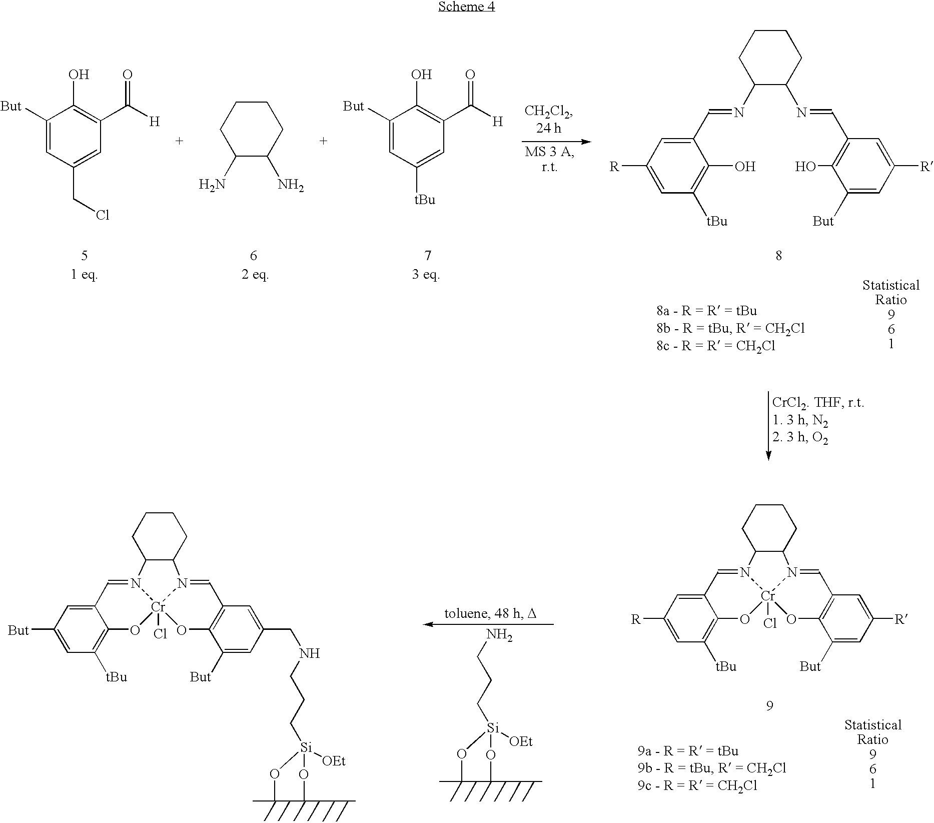

As metal salts with Lewis acid characteristics, the present document includes aluminium trichloride, alkylaluminium compounds, zinc dichloride, iron chlorides, di- and tetrachlorides of tin, titanium tetrachloride and titanium tetralcoxy compounds.

As metal complexes, the present document expressly includes complexes with Schiff bases of aluminium without functionalising and dissolved in any ionic liquid and complexes with Schiff bases of aluminium and chromium duly functionalised with imidazolium substituents or in such a way that they can be anchored to a support. It also includes vinyl monomers of these complexes and polymers and copolymers deriving from them.

The reaction can be carried out in discontinuous, semi-continuous or continuous reactors. The working pressure range lies in the range between atmospheric pressure and 150 bars, the reaction temperature lying between 20° C. and 180° C. The concentration of catalyst is between 0.01 and 30% in moles with respect to the controlling reagent.

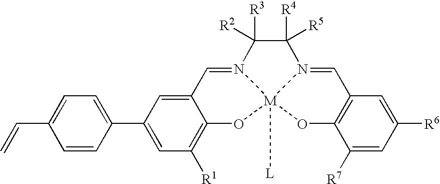

Rⁿ: any alkyl, halogen or alcoxyl group
M: metal cation
L: apical ligand such as chloride or organic base

### EXAMPLES

Described below are some examples of embodiment of the invention:

### Example 1

In a preferred embodiment, (salen)Al(III) chloride (30 mg) is used as catalyst and N-methylimidazol (10 μl) as co-catalyst, dissolved in 1-butyl-3-methylimidazol hexafluorophosphate (0.5 ml) and as reagent ethylene oxide (3.56 mmol), the system being charged in an autoclave which operates with CO₂ and working at a temperature of 80° C. and 100 bars. Under these conditions and after proceeding to recover the reaction products with hexane a conversion of 60% and a selectivity to carbonate of 60% is obtained. The system can be reused after evacuating the ionic liquid in order to eliminate residues of hexane.

Alternatively, the product can be recovered by heating the ionic liquid at 120° C. and condensing the vapours.

### Example 2

The diethyl acetal of formaldehyde (0.5 ml) together with tributylamine and (salen)Al(III) chloride covalently anchored to silica following the sequence of Scheme 4 for the case of chromium (30 mg) are introduced into an autoclave of volume 50 ml which is charged with CO₂ in such way that at 80° C. the system has a pressure of 100 bars. After 6 h of reaction, the autoclave is left to cool and is discharged, with diethyl carbonate being obtained as the reaction product with a yield of 30%.

1-58. (canceled)

59. Method of using a catalytic composition comprising carrying out an insertion reaction of carbon dioxide into an organic compound, wherein said composition comprises:
a first component which is at least one compound of one or more metals from groups 3A, 4A, 5A, 6A, 7A, 8, 1B, 2B, 3B, 4B, and
a second component selected from
at least one ionic liquid which consists of a compound formed by cations and anions and which is a liquid at ambient temperature,
a matrix to which the first component is bound or on which it is supported, and
a combination of the two,
in a method for the insertion of carbon dioxide into an organic compound.

60. Method of use according to claim 59, wherein said organic compound is selected from epoxides, acetals and orthoesters, and combinations of them.

61. Method of use according to claim 59, wherein said first component is a compound selected from a salt, a complex, a vinyl monomer of this metal complex, a polymer of said vinyl monomer, a copolymer of said vinyl monomer and combinations of them.

62. Method of use according to claim 59, wherein the first component is a compound of a metal selected from chromium, cobalt, iron, vanadium and aluminium.

63. Method of use according to claim 59, wherein the first component is a metal complex which has a Schiff base as ligand.

64. Method of use according to claim 59, wherein the first component is a complex of a metal selected from chromium, cobalt, iron, vanadium and aluminium and which has “salen” as ligand.

65. Method of use according to claim 59, wherein said catalytic composition comprises:
a first component which is at least one complex of one or more metals from groups 3A, 4A, 5A, 6A, 7A, 8, 1B, 2B, 3B, 4B,
a second component selected from
at least one ionic liquid which consists of a compound formed by cations and anions and which is a liquid at ambient temperature,
a matrix to which the first component is bound or on which it is supported, and
a combination of the two,
and a basic nucleophilic co-catalytic agent.

66. Method of use according to claim 65, wherein the basic nucleophilic co-catalyst is selected between
a ligand selected between N-methylimidazol, N,N-dimethylaminopyridine and a mixture of both, and
a base selected from tertiary amines, acetates of alkaline cations, acetates of alkaline earth cations, basic carbonates and basic solids.

67. Method of use according to claim 59 or 65, wherein said ionic liquid is a compound in which the cations are organic cations.

68. Method of use according to one of claims 59 or 65, wherein said ionic liquid is a compound in which the cations are organic cations and said organic cations are selected between an imidazolium cation and an imidazolium cation which displays one or more additional alkyl substituents.

69. Method of use according to claim 68, wherein the imidazolium cation displays one or more alkyl substituents selected from methyl, ethyl, propyl, butyl, hexyl, octyl, decyl, dodecyl, tetradecyl, hexadecyl and any of their fluoride derivatives.

70. Method of use according to claim 67, wherein the cation is selected between 1-methylimidazolium, 1-butyl-1-methylimidazolium, 1-ethyl-1-methylimidazolium, 1-butyl-3-ethyl-imidazolium and 1-hexyl-3-methylimidazolium.

71. Method of use according to claim 67, wherein the ionic liquid comprises the cation 1-butyl-1-methyl imidazolium and an anion selected among tetrafluoroborate, phosphate, sulphate, trifluormethanosulphonate, bistrifluoromethanosulphoimidate, chloride, bromide, dialuminium heptachloride and combinations of them.

72. Method of use according to one of claims 59, wherein said ionic liquid is a compound in which the cations are organic cations and said ionic liquid is selected from one or more salts of quaternary ammonium salts, quaternary phosphonium salts and pyridinium salts.

73. Method of use according to claim 59, wherein said composition comprises:
a first component which is at least one compound of one or more metals from groups 3A, 4A, 5A, 6A, 7A, 8, 1B, 2B, 3B, 4B, selected from a salt and a metal complex, and
a second component which is at least one ionic liquid consisting of a compound formed from cations and anions and which is a liquid at ambient temperature.

74. Method of use according to claim 59, wherein said composition comprises:
a first component which is the complex (salen)Al(III) chloride and N-methylimidazol and
a second component which is an ionic liquid, in which the cation is 1-butyl-1-methylimidazolium.

75. Method of use according to claim 59, wherein said composition comprises:
a first component which is at least one compound of one or more metals from groups 3A, 4A, 5A, 6A, 7A, 8, 1B, 2B, 3B, 4B, selected between a salt, a complex and combinations of both, and
a second component which is a matrix selected from a polymeric organic solid and an inorganic solid.

76. Method of use according to one of claims 59 or 75, wherein said matrix is an inorganic solid selected among graphite, activated carbon and an allotropic form of carbon.

77. Method of use according to one of claims 59 or 75, wherein said first component is a metal salt and said second component is a matrix selected from a polymeric organic solid and an inorganic solid.

78. Method of use according to one of claims 59 or 75, wherein said first component is a metal complex and said second component is an inorganic solid to which said metal complex is covalently bound.

79. Method of use according to one of claims 59 or 75, wherein said first component is a metal complex which has a “salen” ligand and said second component is an organic polymer to which said metal complex of “salen” is covalently bound.

80. Method of use according to one of claims 59 or 75, wherein the metal compound is a metal salt which is bound to the matrix electrostatically.

81. Method of use according to claim 75, wherein said first component is a metal complex and wherein said metal complex is covalently anchored to a matrix selected between silica, zeolites and a metal oxide other than zeolites.

82. Method of use according to claim 75, wherein the metal complex or the metal salt is bound to silica, zeolites or other metal oxide by means of electrostatic interactions.

83. Method of use according to claim 59, wherein said composition comprises:
a first component which is at least one compound of one or more metals from groups 3A, 4A, 5A, 6A, 7A, 8, 1B, 2B, 3B, 4B, selected between a salt, a complex and combinations of both, and
a second component which comprises
at least one ionic liquid which consists of a compound formed by cations and anions and which is a liquid at ambient temperature, and
a matrix to which the first component is bound or on which said first component is supported.

84. Method of use of a catalytic composition according to claim 59, wherein said composition comprises:
a first component which is at least one compound of one or more metals from groups 3A, 4A, 5A, 6A, 7A, 8, 1B, 2B, 3B, 4B, selected between a salt and a metal complex, and
a second component which a matrix to which the first component is bound, or on which it is supported and an organic solvent.

85. Method of use of a catalytic composition according to claim 59, wherein the insertion of CO₂ is produced by means of an operation selected from a continuous, semi-continuous and discontinuous reaction.

86. Method of use of a catalytic composition according to claim 59, wherein the insertion of CO₂ takes place in a discontinuous reactor, with an interval of catalyst concentrations lying between 0.01 and 30% by mols of catalyst with respect to said organic compound.

87. Method of use of a catalytic composition according to claim 59, wherein said insertion of CO₂ is a reaction of a starting product selected from the group consisting of ethylene oxide, propylene oxide and styrene oxide which is converted into its corresponding carbonate.

88. Method of use of a catalytic composition according to claim 59, wherein said CO₂ insertion is a reaction of a starting product selected from methyl orthoformate, ethyl orthoformate, propyl orthoformate, isopropyl orthoformate and butyl orthoformate which is converted into the corresponding carbonate according to the equation: 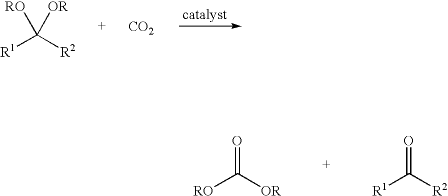
in which the radicals R, R¹ and R² are independently selected between hydrogen, an alkyl residue of 1 to 20 carbon atoms and an aryl residue.

89. Method of use of a catalytic composition according to claim 59, wherein said CO₂ insertion is carried out in a temperature range of between 20° C. and 180° C.

90. Method of use of a catalytic composition according to claim 59, wherein said CO₂ insertion reaction is carried out in a range of between atmospheric pressure and 150 bar.

91. A catalytic composition comprises:
a first component which is at least one compound of one or more metals from groups 3A, 4A, 5A, 6A, 7A, 8, 1B, 2B, 3B, 4B, and
a second component selected from
at least one ionic liquid which consists of a compound formed by cations and anions and which is a liquid at ambient temperature, and
a matrix to which the first component is bound or on which it is supported, together with at least one ionic liquid as defined earlier.

92. A catalytic composition according to claim 91, wherein said first component is a compound selected from a salt, a complex, a vinyl monomer of this metal complex, a polymer of said vinyl monomer, a copolymer of said vinyl monomer and combinations of them.

93. A catalytic composition according to claim 91, wherein the first component is a compound of a metal selected from chromium, cobalt, iron, vanadium and aluminium.

94. A catalytic composition according to claim 91, wherein the first component is a metal complex which has a Schiff base as ligand.

95. A catalytic composition according to claim 91, wherein the first component is a complex with a “salen” ligand of a metal selected from chromium, cobalt, iron, vanadium and aluminium.

96. A catalytic composition according to claim 91, wherein it comprises:
a first component which is at least one complex of one or more metals from groups 3A, 4A, 5A, 6A, 7A, 8, 1B, 2B, 3B, 4B,
a second component which comprises a matrix to which the first component is bound or on which it is supported, together with at least one ionic liquid as defined earlier,
and a basic nucleophilic co-catalytic agent.

97. A catalytic composition according to claim 96, wherein the basic nucleophilic co-catalyst is selected between
a ligand selected from N-methylimidazol, N,N-dimethylaminopyridine and a mixture of both, and
a base selected from tertiary amines, acetates of alkaline cations, acetates of alkaline earth cations, basic carbonates and basic solids.

98. A catalytic composition according to claim 91, wherein said ionic liquid is a compound in which the cations are organic cations.

99. A catalytic composition according to claim 91, wherein said ionic liquid comprises a cation selected between the imidazolium cation and the imidazolium cation with one or more additional alkyl substituents.

100. A catalytic composition according to claim 99, wherein the imidazolium cation displays one or more alkyl substituents selected from methyl, ethyl, propyl, butyl, hexyl, octyl, decyl, dodecyl, tetradecyl, hexadecyl and any of their fluoride derivatives.

101. A catalytic composition according to one of claim 99, wherein the cation is selected from 1-methylimidazolium, 1-butyl-1-methylimidazolium, 1-ethyl-1-methylimidazolium, 1-butyl-3-ethyl-imidazolium and 1-hexyl-3-methylimidazolium.

102. A catalytic composition according to claim 99, wherein the ionic liquid comprises the cation 1-butyl-1-methylimidazolium, and an anion selected from tetrafluoroborate, phosphate, sulphate, trifluormethanosulphonate, bistrifluoromethanosulpho-imidate, chloride, bromide, dialuminium heptachloride and combinations of them.

103. A catalytic composition according to claim 91, wherein said ionic liquid is selected from one or more salts of quaternary ammonium salts, quaternary phosphonium salts and pyridinium salts.

104. A catalytic composition according to claim 91, wherein it comprises:
a first component which is at least one compound of one or more metals from groups 3A, 4A, 5A, 6A, 7A, 8, 1B, 2B, 3B, 4B, selected from a salt and a metal complex, and
a second component which is at least one ionic liquid consisting of a compound formed from cations and anions and which is a liquid at ambient temperature.

105. A catalytic composition according to claim 91, comprising:
a first component which is the complex (salen)Al(III) chloride and N-metlhylimidazol and
a second component which is an ionic liquid, in which the cation is 1-butyl-1-methylimidazolium.

106. A catalytic composition according to claim 105, wherein the complex salen Al(IIII) chloride complex is enantiomerically pure.

107. A catalytic composition according to claim 91, comprising:
a first component which is at least one compound of one or more metals from groups 3A, 4A, 5A, 6A, 7A, 8, 1B, 2B, 3B, 4B, selected between a salt, a complex, and combinations of both, and
a second component which is a matrix selected between a polymeric organic solid and an inorganic solid, together with at least one ionic liquid as defined above.

108. A catalytic composition according to claim 91, wherein said matrix is an inorganic solid selected from graphite, activated carbon and an allotropic form of carbon.

109. A catalytic composition according to claim 91, wherein said first component is a metal salt and said second component is a matrix selected from a polymeric organic solid and an inorganic solid.

110. A catalytic composition according to claim 91, wherein said first component is a metal complex and said second component is an inorganic solid to which said metal complex is covalently bound.

111. A catalytic composition according to claim 91, wherein said first component is a metal complex of “salen”.

112. A catalytic composition according to claim 91, wherein said first component is a metal complex of “salen” and the second component is an organic polymer to which said metal complex of “salen” is covalently bound.

113. A catalytic composition according to claim 91, wherein the metal compound is a metal salt and is bound to the matrix electrostatically.

114. A catalytic composition according to claim 91, wherein said metal complex is covalently anchored to a matrix selected from silica, zeolites or other metal oxide.

115. A catalytic composition according to claim 91, wherein the metal complex or the metal salt is covalently anchored to the silica, zeolites or other metal oxide by means of electrostatic interactions.

116. A catalytic composition according to claim 91, wherein said metal compound is an enantiometrically pure metal complex.
